(19) United States
(12) Patent Application Publication
(10) Pub. No.: US 2018/0140360 A1
(43) Pub. Date: May 24, 2018

(54) ACCURACY TESTING OF ELECTROMAGNETIC DEVICE TRACKING

(71) Applicant: Lucent Medical Systems, Inc., Kirkland, WA (US)

(72) Inventor: Samuel Peter Andreason, Kirkland, WA (US)

(21) Appl. No.: 15/820,001

(22) Filed: Nov. 21, 2017

Related U.S. Application Data
(60) Provisional application No. 62/424,995, filed on Nov. 21, 2016.

(51) Int. Cl. A61B 34/20; G01B 7/00; A61B 5/06

(52) U.S. Cl. CPC A61B 34/20; G01B 7/003; A61B 2034/207; A61B 2034/2051; A61B 2560/0223; A61B 5/062

(57) ABSTRACT

An electronic sensor device test fixture includes an electronic sensor device receiving mechanism arranged to receive an electronic sensor device. The electronic sensor device test fixture also includes a plurality of electromagnetic coils removably or permanently arranged in known positions relative to the electronic sensor device receiving mechanism.

### CROSS-REFERENCE(S) TO RELATED APPLICATION(S)

This application claims the benefit of U.S. Provisional Patent Application No. 62/424,995, filed Nov. 21, 2016, which is hereby incorporated by reference in its entirety to the extent that it does not conflict with the present specification.

### BACKGROUND

### Technical Field

The present disclosure generally relates to testing the accuracy of a sensor that tracks electromagnetic devices. More particularly, but not exclusively, the present disclosure relates to a physical structure having a plurality of electromagnetic devices associated therewith; the position of each electromagnetic device being known, the position of each electromagnetic device being determined by a sensor under test, and each determined position being compared to each known position during a test of the subject sensor.

### Description of the Related Art

In many medical procedures, a medical practitioner accesses an internal cavity of a patient using a medical device. In some cases, the medical practitioner accesses the internal cavity for diagnostic purposes. In other cases, the practitioner accesses the cavity to provide treatment. In still other cases, different therapy is provided.

It is known that in these and in other cases, the medical device may be tracked as it travels or remains stationary within the patient's body. The medical device will carry one or more permanent magnets or electromagnets, which are located by a detection apparatus. To use these types of systems, the medical device is advanced into the body of a patient, and the detection apparatus is moved about the body of the patient. The detection apparatus distinguishes the field strength of the magnet associated with the medical device from the earth's magnetic field and other magnetic energy in order to accurately determine a position of the medical device relative to the detection apparatus.

In these cases, the detection apparatus may be arranged to use three or more sets of magnetic sensors, each magnetic sensor having sensor elements arranged in a known fashion. Each sensor element senses the magnetic field strength generated by the magnet associated with the medical device, and each sensor element provides data indicative of the direction of the magnet in a three-dimensional space. The detection apparatus uses fundamental equations for electricity and magnetism that relate measured magnetic field strength and magnetic field gradient to the location and strength of a magnetic dipole. The detection apparatus uses an iterative process to determine the actual location and orientation of the detected magnet. An initial estimate of the location and orientation of the magnet results in the generation of predicted magnetic field values. The predicted magnetic field values are compared with the actual measured values provided by the magnetic sensors. Based on the difference between the predicted values and the measured values, the detection apparatus estimates a new location of the detected magnet and calculates new predicted magnetic field strength values. This iteration process continues until the predicted values match the measured values within a desired degree of tolerance. At that point, the estimated location matches the actual location within a predetermined degree of tolerance. A two dimensional display provides an indication of the location of the magnet with respect to the housing of the detection apparatus. A depth indicator portion of the display can be used to provide a relative or absolute indication of the depth of the magnet within the patient.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which in and of itself may also be inventive.

### BRIEF SUMMARY

Sensors are produced to track electromagnetic devices. In some cases, the sensors track an electromagnetic device within a patient's body. The electromagnetic device may be stimulated with a low-frequency when the device is within a body in real time. In order to track the electromagnetic devices with an acceptable level of accuracy, one or more calibration procedures are performed with the sensor device. It has been learned that a calibration fixture may be formed having a plurality of electromagnetic coils arranged in known or knowable positions and orientations. As each coil is driven with a low-frequency signal, the generated magnetic field can be detected by the sensor that is being calibrated. Using the known position and orientation of the energized coil, certain numerical values may be produced, which are particular to the sensor that is being calibrated. Subsequently, in normal operation when the sensor device is tracking an electromagnetic device, the produced numerical values (i.e., the calibration values) may be used to correct position and orientation information of electromagnetic device that is being tracked.

The present disclosure describes embodiments and uses of calibration fixtures that may be formed to produce calibration values or other values that may then be applied to improve one or more of the speed, accuracy, range, resistance to interference, or other processing of an electromagnetic device tracking sensor. In some cases, embodiments described in the present disclosure are alternatively or additionally arranged as test fixtures. In at least some of these cases, a test fixture may be used to provide a “pass/fail” indication of a particular sensor device, wherein such pass/fail indication may be associated with the subject sensor device's ability to track one or more electromagnetic coils to a determined level of accuracy.

A first embodiment may be summarized as a test fixture that includes a surface; a plurality of electromagnetic coils, each electromagnetic coil of the plurality arranged in a known or knowable three-dimensional position and orientation relative to any point on the surface; and a receiving mechanism integrated with the surface, the receiving mechanism structured to temporarily receive an electronic sensor device, each reception of the electronic sensor device by the receiving mechanism placing the electronic sensor device in a same three-dimensional position and orientation relative to each electromagnetic coil of the plurality of electromagnetic coils.

In some cases of the first embodiment, the receiving mechanism includes a well formed in the surface, the well having a shape that mates with a shape of a housing of the electronic sensor device. And in some cases of the first embodiment, the test fixture includes a second surface; and a second receiving mechanism integrated with the second surface, the second receiving mechanism structured to temporarily receive the electronic sensor device, each reception of the electronic sensor device by the second receiving mechanism placing the electronic sensor device in a same second three-dimensional position and orientation relative to each electromagnetic coil of the plurality of electromagnetic coils.

In some cases of the first embodiment, the test fixture includes a switching circuit arranged to electrically couple each electromagnetic coil of the plurality of electromagnetic coils to a signal driving circuit associated with the electronic sensor device. In some of these cases, the switching circuit is arranged to electrically couple each electromagnetic coil of the plurality of electromagnetic coils to the signal driving circuit associated with the electronic sensor device in a determined sequence. In some of these cases, the switching circuit is arranged to electrically couple two electromagnetic coils of the plurality of electromagnetic coils to the signal driving circuit associated with the electronic sensor device concurrently. In others of these cases, the test fixture also includes a plurality of drive signal conduits, each drive signal conduit associated with a different one of the plurality of electromagnetic coils, the plurality of drive signal conduits arranged to facilitate the passage of drive signals to its associated electromagnetic coil. And in still others of these cases, the test fixture includes at least one wireless transceiver arranged to facilitate the passage of drive signals to the plurality of electromagnetic coils.

In some cases of the first embodiment, the test fixture includes a plurality of support structures physically coupled to the test fixture, at least one electromagnetic coil of the plurality of electromagnetic coils integrated with each support structure. In some of these cases, individual ones of the plurality of support structures may be repositioned.

A second embodiment may be summarized as an electronic sensor device test fixture that includes an electronic sensor device receiving mechanism arranged to receive an electronic sensor device; and a plurality of electromagnetic coils arranged in known positions relative to the electronic sensor device receiving mechanism.

In some cases of the second embodiment, the plurality of electromagnetic coils are removably arranged in known positions relative to the electronic sensor device receiving mechanism. In some cases, the plurality of electromagnetic coils are permanently arranged in known positions relative to the electronic sensor device receiving mechanism. In some cases, the electronic sensor device receiving mechanism includes at least one registration mechanism to align the electronic sensor device. And in still some other cases of the second embodiment, the electronic sensor device test fixture includes a base unit, the base unit having the electronic sensor device receiving mechanism integrated therein; and a plurality of support structures affixed to the base unit, at least some of the plurality of support structures having at least some of the plurality of electromagnetic coils integrated therein.

A third embodiment may be summarized as a test fixture method that includes acts of coupling an electronic sensor device to a test fixture, the test fixture having a surface and a plurality of electromagnetic coils, each electromagnetic coil of the plurality arranged in a known or knowable three-dimensional position and orientation relative to any point on the surface; directing at least one drive signal to at least one electromagnetic coil of the plurality of electromagnetic coils to energize the respective at least one electromagnetic coil; sensing a magnetic field generated about each energized electromagnetic coil; and generating position and orientation information associated with each energized electromagnetic coil.

In some cases of the third embodiment, the test fixture method includes sequentially directing a plurality of drive signals to various ones of the plurality of electromagnetic coils; and capturing the generated position and orientation information associated with each energized electromagnetic coil. And in some of these cases, the test fixture method includes storing the generated position and orientation information in the electronic sensor device as calibration information.

In some cases of the third embodiment, the magnetic field formed about each energized each coil has characteristics directed by characteristics of the at least one drive signal. In some of these or other cases, at least two electromagnetic coils of the plurality of electromagnetic coils are concurrently energized.

This Brief Summary has been provided to introduce certain concepts in a simplified form that are further described in detail below in the Detailed Description. Except where otherwise expressly stated, the Brief Summary does not identify key or essential features of the claimed subject matter, nor is it intended to limit the scope of the claimed subject matter.

### BRIEF DESCRIPTION OF THE SEVERAL VIEWS OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIGS. 1A-1D are an electronic sensor device test fixture embodiment;

FIG. 2 is another electronic sensor device test fixture embodiment;

FIG. 3 is a flow control diagram representing exemplary operations of an electronic sensor device test fixture.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with computing systems including client and server computing systems, as well as networks have not been shown or described in detail to avoid unnecessarily obscuring descriptions of the embodiments.

In many low-frequency electromagnetic tracking systems, an electromagnetic medical device such as a stylet is tracked within the body of a patient. In such systems, one or more electromagnetic coils are integrated with the medical device. The one or more electromagnetic coils are driven using a wired or wireless circuit that instantiates a low-frequency drive signal through the respective coil, and the low-frequency drive signal causes a magnetic field to form about the respective coil.

The magnetic field formed about each coil has particular characteristics (i.e., properties) that are influenced or otherwise directed by the characteristics (i.e., properties) of the drive signal. For example, based on certain drive signal characteristics such as voltage, current, frequency, phase, polarity, and the like, the generated magnetic field will be formed with certain correlated characteristics such as flux, magnitude (i.e., strength), duration, polarity, direction, orientation, and the like.

In addition, other characteristics of the generated magnetic field are influenced or otherwise directed by the characteristics (i.e., properties) of the coil. For example, the size, shape, materials, number of coils, density of coil windings, one or more diameters of formed coils, orientation in three-dimensional space, surrounding materials and structures, and the like may all influence the characteristics of a generated magnetic field.

In the systems described herein, a medical device that bears at least one electromagnetic coil is tracked using an electronic sensor device. For example, as the medical device is moved within the body of a patient, known drive signals are applied to the one or more coils integrated with the medical device. When the drive signals are applied, the electronic sensor device detects the one or more generated magnetic fields. Using a particular algorithm or set of algorithms, the electronic sensor device also generates and presents a representative position of the medical device through an associated user interface (e.g., a display, an audio device, a tactile feedback device, and the like).

In order to use the electronic sensor device to accurately track a medical device that bears at least one electromagnetic coil, the electronic sensor device is calibrated. The calibration procedure may be performed during manufacture of an electronic sensor device, during testing of an electronic sensor device, in the field in association with normal use or maintenance of an electronic sensor device, or at other times. The testing is performed to determine whether or not the electronic sensor device can track an electromagnetic device with acceptable accuracy.

In production, for example, assembled electronic sensor devices are tested and calibrated using a calibration fixture. One traditional approach used in the past to test and calibrate certain electronic sensor devices was to removably affix an electronic sensor device in a known position. After affixing the electronic sensor device, a medical device having a magnet formed thereon was moved relative to the electronic sensor device in a controlled fashion through a series of points. The points were conventionally controlled by software (e.g., manually moved according to a computer-directed user interface, robotically, or in a like fashion), which moved the medical device through a series of X-Y-Z-rotation linear translation stages. Since the positions that electronic sensor device generates for the medical device are “known,” the electronic sensor device may be calibrated by setting values, ranges, or other parameters in such a way that the electronic sensor device reports the position with acceptable accuracy. The values, ranges, or other parameters may be zeroing values, weighting factors, offsets, rounding parameters, or the like.

The traditional approach provides a system that is flexible in testing and calibrating a wide range of medical device positions and orientations, but the traditional approach also has undesirable limitations. For example, the traditional approach is limited in that it does not have the ability to test all rotations of the medical device relative to the sensor because the medical device is confined to a single rotational plane. While more fully functional robot-arm style systems are more flexible (i.e., these systems may have multiple rotational planes), these robot-arm style systems have moving parts that require their own frequent calibration, wear out, operate slowly, cause interference, and introduce other undesirable limitations.

A new calibration and testing system is disclosed herein that does not have the same undesirable limitations of the traditional approach.

Due to the nature of the electronic sensor devices that can track electromagnetic devices, the structures described herein pre-position a plurality of electromagnetic devices within range of an electronic sensor device under test. The electromagnetic devices (i.e., “coils”) can be placed in any conceivable position, distance, orientation, and the like. The electromagnetic devices may be in fixed positions or they may be in removable or adjustable positions. The electromagnetic devices may have fixed orientation or they may be rotatable about one or more axes.

In some cases, the electronic sensor device directs the phase and frequency with which an electromagnetic device is driven. In a wireless electronic sensor device, an identifier of each electromagnetic device may also be directed by the electronic sensor device. The electronic sensor device system can then electrically determine and direct which electromagnetic device is being driven. For example, the electronic sensor device may drive each electromagnetic device in a known sequence to test various positions. Known positions, angles, orientations, and the like of the electromagnetic devices, relative to each other or relative to the electronic sensor device, may then be compared to the positions generated by the electronic sensor device. In this way, the accuracy of each electronic sensor device may be determined, and one or more calibration values may be generated and stored for later use by the electronic sensor device.

In this new system, since there are no moving parts, the need for frequent calibration is reduced or even eliminated. In addition, the speed of testing (e.g., calibrating) each electronic sensor device may be improved because the procedure may complete as quickly as the electronic sensor device can identify, lock on, and track each electromagnetic device. What's more, a substantial number of possible electromagnetic device positions can be tested.

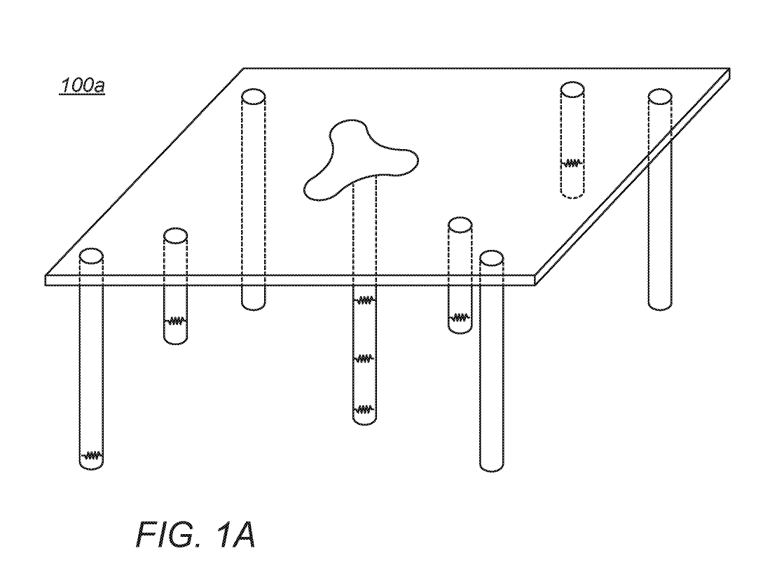

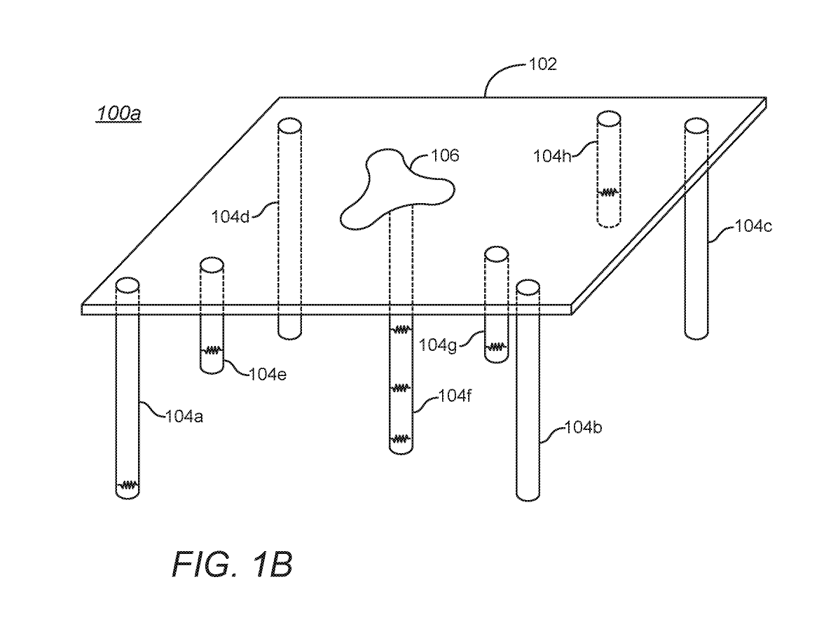

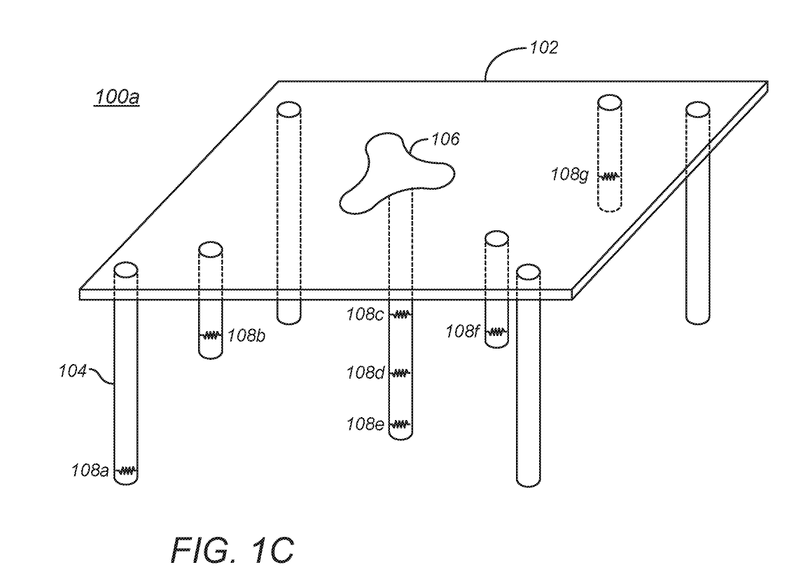

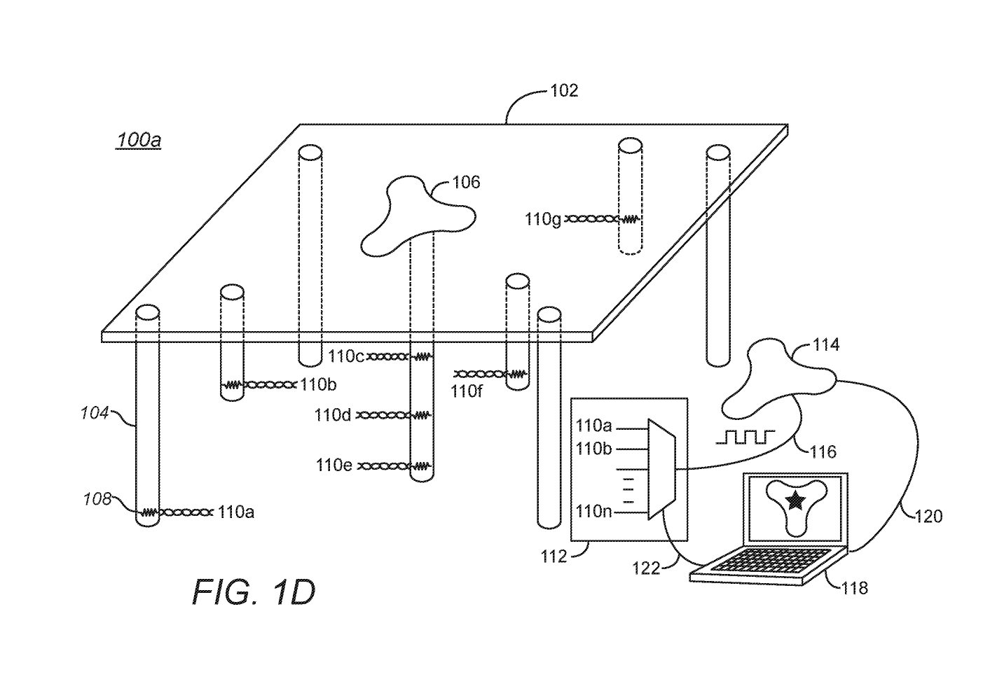

FIGS. 1A-1D are an electronic sensor device test fixture embodiment 100a. Embodiments of the test fixture 100a may be used to test an electronic sensor device, calibrate an electronic sensor device, certify or otherwise validate the accuracy of an electronic sensor device, or perform some other action associated with an electronic sensor device. In use, the test fixture 100a receives an electronic sensor device, and one or more electromagnetic coils are energized serially, concurrently, or in some desirable order. The electromagnetic coils are integrated or otherwise associated with the test fixture 100a in known positions relative to the electronic sensor device. Concurrent with the energizing of certain electromagnetic coils, the electronic sensor device is operated to determine the position of said coils. By comparing the known position of a coil with a generated position of the coil produced by the electronic sensor device, the electronic sensor device may be tested, validated, calibrated, or the like.

In FIG. 1A, the electronic sensor device test fixture 100a is generally formed having an electronic sensor device receiving mechanism arranged to receive an electronic sensor device, and a plurality of electromagnetic coils removably or permanently arranged in known positions relative to the electronic sensor device receiving mechanism.

The test fixture 100a of FIG. 1A is arranged having a generally flat surface on or in which the electronic sensor device receiving mechanism is formed, but other embodiments are contemplated. In some cases, the electronic sensor device receiving mechanism may be formed on a convex surface, a concave surface, an irregular surface, or no surface at all. In embodiments of the present invention, an electronic sensor device under test may be repeatably positioned in, on, or in some other association such that the electronic sensor device under test is in a known linear and angular orientation to the electromagnetic coils that will be energized.

The test fixture 100a may be generally formed from one or more types of metal, one or more types of plastic, one or more types of wood, or some other solid substance alone or in a cooperative combination. For example, in some cases, the test fixture 100a is formed substantially from a non-metallic thermoplastic resin (e.g., an acrylic material).

A test fixture 100a embodiment may have one or more support structures, such as legs, walls, pins, or the like. The support structures may be permanently or removably attached to the test fixture 100a. The support structures may be arranged to provide spatial diversity of the electronic sensor device under test from one or more electromagnetic coils. In the alternative, or in addition, the support structures may be arranged to facilitate an improved usability of the test fixture 100a. In some cases, the test fixture 100a does not have any support structures.

In FIG. 1B, the electronic sensor device test fixture 100a includes a base unit 102 formed as a first platform. A plurality of support structures 104, which are individually referenced as 104a-104h, are integrated, coupled, or otherwise arranged in unity with the base unit 102. In some cases, the support structures 104 are arranged as footings that support the base unit 102 above a work table or another underlying structure. In some cases, the support structures 104 are arranged to accommodate one or more electromagnetic coils. In still other cases, the support structures provide both support of the base unit 102 and accommodation of one or more electromagnetic coils.

An electronic sensor device receiving mechanism 106 is assembled, integrated, or otherwise formed in cooperation with the base unit 102. In some cases, the electronic sensor device receiving mechanism 106 is a hollowed receptacle to receive an electronic sensor device. In these and other embodiments, the hollowed receptacle is formed having a shape that will tightly follow, mate, or otherwise receive a portion of the housing of an electronic sensor device. In this way, the electronic sensor device under test may be repeatably placed in the test fixture 100a in a known position and orientation.

The electronic sensor device receiving mechanism 106 may be further formed with other registration features to facilitate the repeatable placement of the electronic sensor device in the electronic sensor device receiving mechanism 106. For example, the electronic sensor device receiving mechanism 106 may include pins, straps, clamps, registration structures (e.g., protrusions, apertures, valleys, and the like), alignment markings, alignment text, friction surfaces, and the like.

The test fixture 100a illustrated in FIG. 1C identifies a plurality of electromagnetic coils 108, which are individually referenced as 108a-108g. A test fixture 100a may have a single electromagnetic coil 108 or any number of electromagnetic coils 108. In some cases, electromagnetic coils 108 are integrated with support structures 104, in some cases, electromagnetic coils 108 are integrated with base unit 102, and in these or still other cases, electromagnetic coils 108 are assembled in the test fixture 100a in some other way.

Electromagnetic coils 108 may be removably or permanently joined into the test fixture 100a. For example, the electromagnetic coils 108 may be glued, embedded (e.g., encased in a liquid plastic that later hardens), bolted, screwed, riveted, or joined in some other way. Once an electromagnetic coil 108 is placed in the test fixture 100a, the position of the electromagnetic coil 108 should not easily change, and the known position of the electromagnetic coil 108 should be determined. In this way, when an electronic sensor device under test determines a position of the electromagnetic coil 108, the determined position may be compared to the known position of the electromagnetic coil.

In some embodiments, several electromagnetic coils 108 are positioned in close proximity to each other. Such placement may be used to help determine an acceptable accuracy of an electronic sensor device under test. In these or in other cases, electromagnetic coils 108 may be positioned having first orientation, second orientation, or some other orientation. For example, a first electromagnetic coil 108 may be arranged orthogonal to a second electromagnetic coil 108. In another example, a third electromagnetic coil 108 may be arranged parallel to a fourth electromagnetic coil 108. Other arrangements, orientations, and positions of electromagnetic coils 108 are contemplated.

One or more of the support structures 104 may be moved, rearranged, or otherwise repositioned. The support structures 104 may be permanently or removably affixed to the base unit 102 as described herein. In this way, a support structure that carries one or more electromagnetic coils 108 may be desirably positioned relative to the electronic sensor device receiving mechanism 106. Though the support structures 104 and electromagnetic coils 108 are generally illustrated a “below” the base unit 102 and below the electronic sensor device receiving mechanism 106, other positions and orientations are contemplated. For example, in some cases, one or more support structures 104, one or more electromagnetic coils 108, or one or more support structures 104 and one or more electromagnetic coils 108 are formed “above” the base unit 102, “above” the electronic sensor device receiving mechanism 106, or in some other configuration.

In some cases, the base unit 102 has a surface area of about 400 square inches (in²). For example, the base unit 102 may be formed at about 16 inches×24 inches. In other embodiments, the base unit 102 has a surface area larger than 400 in² or smaller than 400 in². In some embodiments, the longest support structures 104 are about 24 inches. In other embodiments, the longest support structures are longer or shorter than 24 inches.

In FIG. 1D, each electromagnetic coil 108 of the electronic sensor device test fixture 100a has coupled thereto a first drive signal conduit 110, which are individually referenced as 110a-110g. In some cases, the first drive signal conduits 110 are electrically connected to a switching circuit 112, which may include one or more demultiplexor circuits, one or more multiplexor circuits, or some other electronic switch configuration. In these cases, the first drive signal conduit 110 may include a twisted pair of electrical wires, one or more electrical connectors, shielded jacketing, or other features. In other cases, such as in cases where electromagnetic devices are wirelessly directed, the first drive signal conduit 110 may include a wireless transceiver circuit that is further arranged to generate drive signals for an associated electromagnetic coil.

An electronic sensor device 114 is illustrated in the FIG. 1D. The electronic sensor device 114 is an electronic sensor device under test. The testing may include accuracy testing, operational testing, burn-in, calibration, or other testing. During testing operations, the electronic sensor device 114 is mechanically coupled to the electronic sensor device receiving mechanism 106. For example, the electronic sensor device receiving mechanism 106 is formed as a receptacle whose features are arranged to mate with a portion of the housing of electronic sensor device 114. In this way, a user may physically place the electronic sensor device 114 into the suitably shaped and sized electronic sensor device receiving mechanism 106. When properly and firmly seated, the electronic sensor device 114 will have little or no motion. In this way, the electronic sensor device 114 may be repeatably placed in the test fixture 110a such that any electronic sensor device 114 placed in the test fixture 100a will be firmly positioned relative to each electromagnetic coil 108 in a known, repeatable way. In some embodiments, the electronic sensor device 114 is electrically coupled to the switching circuit 112 by a second drive signal conduit 116. The second drive signal conduit 116 may include a twisted pair of electrical conductors. When so directed, the switching circuit 112 may electrically connect the electronic sensor device 114 to one or more electromagnetic coils 108 via the first drive signal conduit 110 and second drive signal conduit 116.

The electronic sensor device 114 is coupled to a computing device 118 via a first control conduit 120. The first control conduit 120 may be a wired conduit or a wireless conduit. For example, the first control conduit 120 may follow a universal serial bus (USB) protocol, an IEEE communications protocol (e.g., RS-232, RS-485, and the like), or some other wired protocol. As another example, the first control conduit 120 may follow a WiFi protocol (e.g., IEEI 802.11) or some other wireless protocol. In some cases, information is communicated via the first control conduit 120. In these and in other cases, power may also be passed via the first control conduit 120.

The computing device 118 may be a desktop computer, a laptop computer, a tablet computer, a smartphone, or some other computing device. In some cases, the computing device 118 is merely an optional electronic user interface device (e.g., display, keyboard, mouse, tactile device, audio device, and the like). In these cases, one or more algorithms to carry out testing, calibration, and the like may be embodied in the electronic sensor device 114.

The computing device 118 of FIG. 1D is communicatively coupled to the switching circuit 112 via a second control conduit 122. In this way, the computing device 118 is arranged to intelligently direct the switching circuit 112 to electrically couple a drive signal generated or otherwise directed by the electronic sensor device 114 to one or more electromagnetic coils 108. Coupling a drive signal, which may be passed via the first drive signal conduit 110 and the second drive signal conduit 116, to an electromagnetic coil 108 will energize the electromagnetic coil 108 and cause a magnetic field to form about the energized electromagnetic coil 108. The generated magnetic field may be sensed by the electronic sensor device 114, and a position and orientation of the energized electromagnetic coil 108 may be determined.

In some cases, the computing device 118 determines the position and orientation of the electromagnetic coil 108 in free space. In some cases, the computing device 118 determines the position and orientation of the electromagnetic coil 108 relative to a baseline such as a point on the test fixture 100a. The determined position may be an actual position or a predicted position. In some cases, the computing device produces information representing the position and orientation of an electromagnetic coil 108, and the information may be presented on a user interface (e.g., display, speaker, and the like). Such information may be used by a user to determine whether the electronic sensor device 114 is operating with acceptable accuracy. In addition, or in the alternative, such information may be used to generate calibration parameters that will later be used by the electronic sensor device 114.

In some cases, the drive signal is coupled to a single electromagnetic coil 108. In some cases, the drive signal is sequentially coupled to a plurality of electromagnetic coils 108, one-at-a-time. In some cases, a first drive signal is coupled to a first electromagnetic coil 108 and concurrently, a second drive signal is coupled to a second electromagnetic coil 108. Accordingly, it is contemplated that any number of drive signals may be coupled to any number of electromagnetic coils 108 individually or concurrently. The drive signals in some cases are low frequency drive signals. For example, the drive signals may be at or about 320 Hz, 330 Hz, or some other frequency that is below 1000 Hz and preferably below 500 Hz. In other cases, a drive signal may be above 1000 Hz and below about 10,000 Hz.

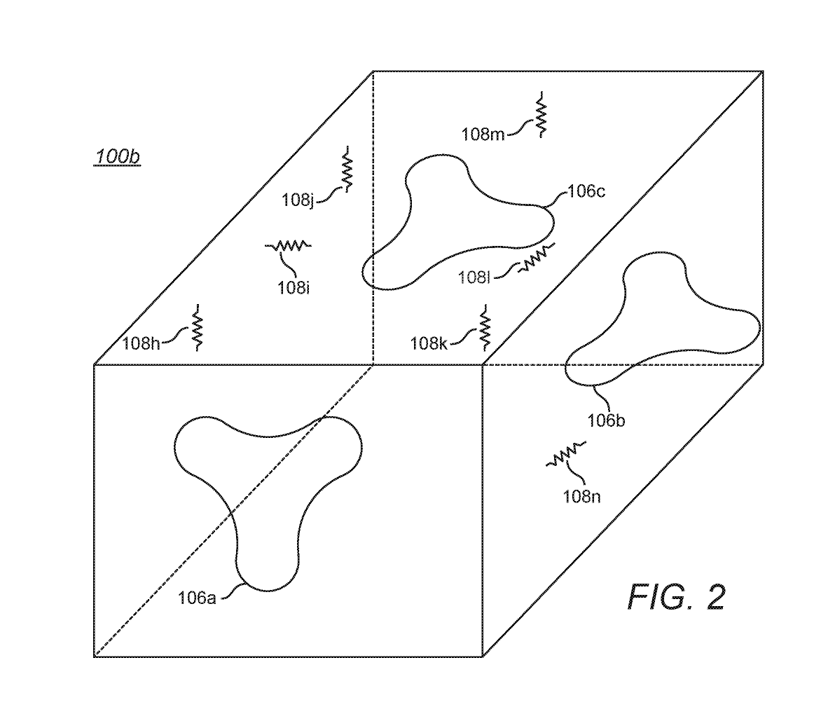

FIG. 2 is another electronic sensor device test fixture embodiment 100b. The test fixture 100b of FIG. 2 may be formed as a solid structure, a caged structure, or a structure composed within some other type of frame. The test fixture 100b may be formed having any dimensions. For example, in the test fixture 100b, a length, a width, and/or a depth may be about 10 inches, 24 inches, 36 inches, 100 inches, or some other size.

The test fixture 100b is arranged with a plurality of electromagnetic coils 108, which are referenced in FIG. 2 as 108h-108n. The electromagnetic coils 108 may be permanently or removably affixed in any desirable position and orientation as described in the present disclosure.

The test fixture 100b includes a plurality of electronic sensor device receiving mechanisms 106a, 106b, 106c. In this way, the test fixture may be adapted to concurrently receive a plurality of electronic sensor devices 114. In this way, a single test fixture 100b may be adapted to test a wide range of configurations.

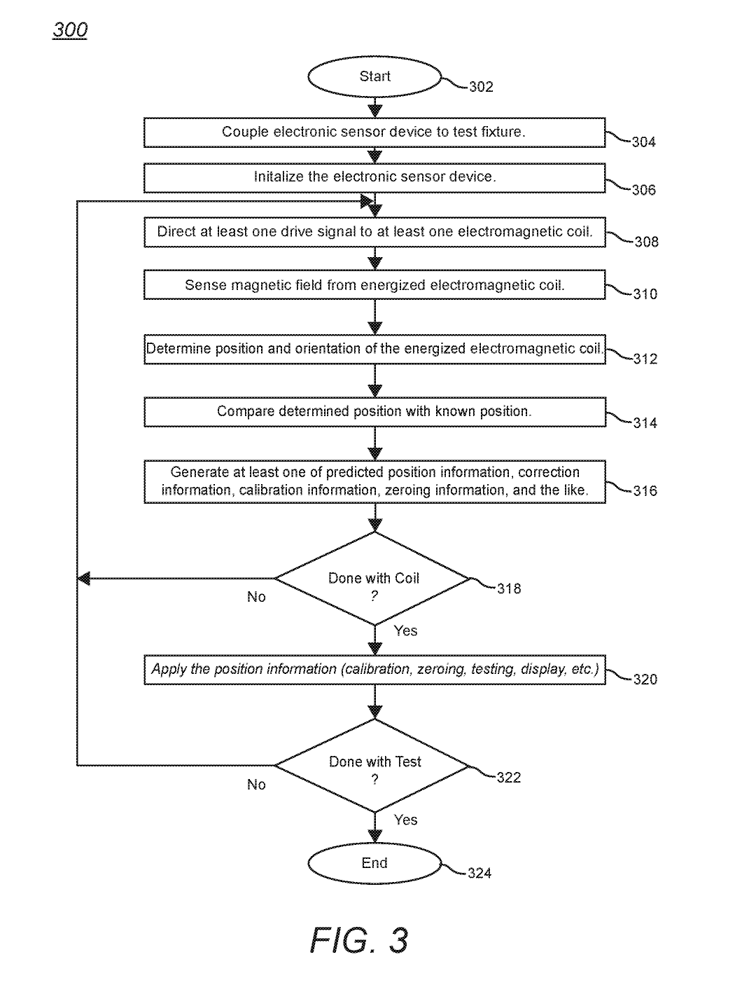

FIG. 3 is a flow control diagram representing exemplary operations of an electronic sensor device test fixture 300. Generally speaking, the exemplary operations to collect calibration data, zeroing data, accuracy data, or the like include first directing a drive signal to an electromagnetic coil, and then recording the magnetic fields about the electromagnetic coil using all of the subject sensors of an electronic sensor device. The drive signal is then directed to a “next” electromagnetic coil for recording the magnetic fields of the “next” electromagnetic coil, and the process is repeated until all of the subject electromagnetic coils have been driven and the results recorded. Calibration information for any or all of the sensors may then be generated. The calibration information may be associated with any arrangement of sensitivity, sensor positions, sensor orientation, board orientation/position, and the like, and the generated calibration information may then be applied to the sensing system of the electronic sensor device.

Subsequently, or at another time, the accuracy of any electronic sensor device may be tested using the test fixture 100a, 100b. In these accuracy testing cases, a first drive signal is directed to a first electromagnetic coil, and the position generated by the electronic sensor device is recorded. The generated position data may then be compared to known position data, and if the comparison result is outside an accepted threshold (e.g., tolerance), then a “FAIL” condition is recognized. Alternatively, or next, each other electromagnetic coil may be driven and generated magnetic field data recorded, and the generated position data associated with each electromagnetic coil may be compared to known or otherwise accepted data. If the generated position data for each electromagnetic coil is within an accepted threshold, then a “PASS” condition is recognized.

With respect to the “known position data” referred to herein, such sensor sensitivity data, orientation data, and other data may be desirably derived from a Helmholtz cage or in some other manner. In this way, the processes described herein may be focused to solve for sensor positions, board position, and the like, which may improve the accuracy of the results.

Turning more specifically to the exemplary operations of an electronic sensor device text fixture 300 in FIG. 3, processing in an electronic sensor device 114, a computing device 118, or both an electronic sensor device 114, and a computing device 118 begins at 302.

At 304, the electronic sensor device 114 is coupled to a test fixture such as test fixture 100a, test fixture 100b, or another test fixture. The electronic sensor device 114 may be snugly fit into an electronic sensor device receiving mechanism 106. In some embodiments, the electronic sensor device 114 is clamped, clipped, strapped, or otherwise affixed in the test fixture, and at 306, the electronic sensor device 114 is initialized.

In some cases, the electronic sensor device 114 is initialized into a specific test program. The test program may be a pre-calibration program, a calibration program, a zeroing program, a data collection program, or some other type of test program. For example, in some cases, a pre-calibration program includes collecting a large volume of magnetic field data for a plurality of electromagnetic coils 108 prior to generating calibration information. In other cases, the electronic sensor device 114 operates in a normal mode that is not especially directed toward a test routine. The initialization may include storing initial position information, orientation information, sensitivity information, and other such information for each sensor integrated in a particular electronic sensor device. Subsequently, such sensor information and any one or more data points associated with such sensor information (e.g., position information, orientation information, sensitivity information, or the like) may be updated during the calibration or other procedures.

At 308, one or both of the electronic sensor device 114 and the computing device 118 direct at least one drive signal to at least one electromagnetic coil 108. In some cases, the drive signal is a signal such as a square wave that oscillates below 10,000 Hz, preferably below 500 Hz, such as at 320 Hz or 330 Hz. In association with forming, enabling, passing, or otherwise directing the drive signal, at least one electromagnetic coil 108 is selected by appropriate operation of the switching circuit 112. In some cases, appropriate operation of the switching circuit 112 includes passing demultiplexor selection information from a computing device 118 to the switching circuit 112 via a second control conduit. The demultiplexor selection information may be operative to permit passage of the drive signal through a demultiplexor to one or more selected electromagnetic coils such as one or more of electromagnetic coils 108a-108g.

Processing advances to 310 where a magnetic field created about a selected and energized electromagnetic coil 108 is sensed with the electronic sensor device 114 that is under test. Based on magnetic information sensed by the electronic sensor device 114, position and orientation of the selected energized electromagnetic coil 108 may be predicted or otherwise determined at 312.

At 314, the determined position of the selected and energized electromagnetic coil 108 is compared with a known position of the selected and energized electromagnetic coil 108. In some cases, the known position of each electromagnetic coil 108 may be determined with visual assistance. For example, a laser measuring device (not shown) or some other electronic device may be used to measure, calculate, or otherwise determine the distance, angle, orientation, and other such information relative to a baseline. The baseline may be a fixed position on the test fixture 100a, 100b, such as particular point of the electronic sensor device receiving mechanism 106.

Based on the comparison information, certain information associated with the particular electromagnetic coil 108 may be determined at 316 by the electronic sensor device 114, the computing device 118, or some combination of the two. The information may include predicted position information, position correction information, angular correction information, orientation correction information, calibration information, zeroing information, or some other type of information.

At 318, processing determines whether processing for the one or more selected electromagnetic coils is complete. For example, in cases where a plurality of electromagnetic coils 108 are sequenced or energized in some other order, the processing of the coils may be based on a time-slice system, a round-robin system, a least recently used (LRU) system, or some other system. In these or in other schemes, each electromagnetic coil 108 may be energized one time or a plurality of times. In some embodiments, certain electromagnetic coils 108 are energized more or less frequently than other electromagnetic coils 108. If operations associated with the selected electromagnetic coil 108 are not complete, processing returns to 308. Alternatively, if operations associated with the selected electromagnetic coil 108 are complete, processing advances to 320.

At 320, some or all of the determined position information is applied. Such application may create one or more parameters associated with calibration, zeroing, or some other operations. Application of the determined position information may also or alternatively include displaying some or all of the information. The determined information may be stored locally or on another computing device such as a web based computing device.

If operations of the test algorithm are not completed at 322, processing returns to 308 where another electromagnetic coil 108 is selected. In contrast, if operations of the test algorithm are completed, processing falls to 324 wherein the operations end.

The exemplary operations of an electronic sensor device text fixture 300 described with respect to FIG. 3 may be flexibly performed in the order shown or in some other order. In addition, the processes may include one or more loops, recalculations, and re-collections of additional data to further collect and refine generated calibration values. For example, in some cases, certain calibration data may also be based on reported positions, sensed fields, particular timing information or other types of processing. In some cases, data from a plurality of electromagnetic coils will be iteratively collected and re-collected to generate a selected sufficient level of information prior to generating the associated corrections values, calibration values, zeroing values, and the like.

In the present disclosure, the terms “calibration,” “calibration factors,” “calibration information,” “calibration values,” “zeroing value,” “zeroing factors,” “zeroing factors,” and other such and related terms are used inclusively to mean any or all numerical data, control data, and other data used by one or more electronic devices, one or more algorithms, and in addition or in the alternative, one or more combinations of electronic devices and algorithms to generate positional information associated with a particular electromagnetic coil, wherein the positional information may include location, angle, orientation, motion, size, shape, materials of composition, magnetic field strength, device identification, and the like. Without limitation, the subject terms may include, for example, parameters to initialize and operate magnetic sensors, analog-to-digital counters, amplifiers, drive circuits, mathematic circuits, timing circuits, and other circuits; numerical data such as scale factors, weighting factors, multipliers, default values, pre-load values, time values, offsets, gains, cross-axis terms, and other values; and control information to direct the operations of said electronic circuits, processors, memory devices, communication devices, and other devices. To this end, at least some of the fixture embodiments (e.g., calibration fixtures, test device fixtures, etc.) described herein are multi dipole-coil test fixtures that enable a user to perform both initial electronic sensor device calibration and post-calibration accuracy testing. In these cases, particularly in initial calibration cases, specific information associated with the fixture such as the locations and dipole-fields of individual small source coils and collections of source coils, is known with acceptable accuracy. This information may be independent and associated in free space. Alternatively, this information may be referenced to one or more other structures, orientations, and the like such as with reference to an electronic sensor device, such as with reference between different electromagnetic coils, or in some other referential arrangement. In some cases, particular details of the coils and relationships between coils and sensor structures associated with certain ones of the fixture embodiments are particularly characterized by generating or map or other like set of data using a single three-axis electronic sensor device moved through free space.

When considering tracking an electromagnetic device with acceptable accuracy, the acceptable accuracy may be based on several factors including the implementation, the size of the electromagnetic coil, the distance of the coil being tracked from the electronic sensor device, the procedure being performed, and other factors.

In some cases, acceptable accuracy is a volumetric term. For example, a position of a coil or its associated medical device may be determined with acceptable accuracy if the true position of the device being tracked is within a selected distance of the position that is generated and represented by the electronic sensor device. For example, considering the true position of a single point of the electromagnetic device being tracked, a hypothetical sphere is formed about the single point, and the radius of the hypothetical sphere is identified as the acceptable accuracy. In this way, when the electronic sensor device generates a representative position of any point on the electromagnetic device being tracked, the representative position of that point must fall within the hypothetical sphere formed about the corresponding point. As another example, a hypothetical “bubble” around the electronic device being tracked may be envisioned. The surface of the hypothetical bubble is determined at the selected distance outward from the surface of the electromagnetic device. In this way, when the electronic sensor device generates a representative position of the electromagnetic device being tracked, the represented position must fall within the hypothetical bubble.

In some cases, an acceptable accuracy is achieved when a generated position of a tracked electromagnetic device is represented within one (1) centimeter (cm) of the true position of the tracked electromagnetic device. In other cases, the selected distance of acceptable accuracy is 1 millimeter (mm), 5 mm, 2 cm, or some other selected distance.

In some cases, an acceptable accuracy also includes a range or level of angular accuracy. Along the lines of “acceptable accuracy” as a volumetric term, angular accuracy involves similar concepts as applied in angle space as a surface term. For example, in some cases, a system may specify an orientation accuracy requirement within 1 degree, within 10 degrees, or within some other angular dimension. Angular accuracy may be specified in terms of a selected coordinate system (e.g., x, y, z, theta, phi), which is a spherical coordinate system with regards to spatial orientation. Specifying accuracy in such a coordinate system may in some cases be as simple as defining acceptable angular accuracy such that theta and phi are at or within 1 degree of true. However, in other cases, acceptable angular accuracy determinations include additional parametric terms such as poles, wherein crossing a particular pole causes an associated coordinate to flip sign.

In the foregoing description, certain specific details are set forth to provide a thorough understanding of various disclosed embodiments. However, one skilled in the relevant art will recognize that embodiments may be practiced without one or more of these specific details, or with other methods, components, materials, etc. In other instances, well-known structures associated with electronic and computing systems including client and server computing systems, as well as networks have not been shown or described in detail to avoid unnecessarily obscuring descriptions of the embodiments.

Certain words and phrases used in the specification are set forth as follows. The terms “include” and “comprise,” as well as derivatives thereof, mean inclusion without limitation. The term “or,” is inclusive, meaning and/or. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like. The term “controller” means any device, system, or part thereof that controls at least one operation, such a device may be implemented in hardware, firmware, or software, or some combination of at least two of the same. The functionality associated with any particular controller may be centralized or distributed, whether locally or remotely. Other definitions of certain words and phrases may be provided within this patent document. Those of ordinary skill in the art will understand that in many, if not most instances, such definitions apply to prior as well as future uses of such defined words and phrases.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content and context clearly dictates otherwise. It should also be noted that the conjunctive terms, “and” and “or” are generally employed in the broadest sense to include “and/or” unless the content and context clearly dictates inclusivity or exclusivity as the case may be. In addition, the composition of “and” and “or” when recited herein as “and/or” is intended to encompass an embodiment that includes all of the associated items or ideas and one or more other alternative embodiments that include fewer than all of the associated items or ideas.

The headings and Abstract of the Disclosure provided herein are for convenience only and do not limit or interpret the scope or meaning of the embodiments.

The various embodiments described above can be combined to provide further embodiments. Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A test fixture, comprising:
a surface;
a plurality of electromagnetic coils, each electromagnetic coil of the plurality arranged in a known or knowable three-dimensional position and orientation relative to any point on the surface; and
a receiving mechanism integrated with the surface, the receiving mechanism structured to temporarily receive an electronic sensor device, each reception of the electronic sensor device by the receiving mechanism placing the electronic sensor device in a same three-dimensional position and orientation relative to each electromagnetic coil of the plurality of electromagnetic coils.

2. A test fixture according to claim 1, wherein the receiving mechanism comprises:
a well formed in the surface, the well having a shape that mates with a shape of a housing of the electronic sensor device.

3. A test fixture according to claim 1, comprising:
a second surface; and
a second receiving mechanism integrated with the second surface, the second receiving mechanism structured to temporarily receive the electronic sensor device, each reception of the electronic sensor device by the second receiving mechanism placing the electronic sensor device in a same second three-dimensional position and orientation relative to each electromagnetic coil of the plurality of electromagnetic coils.

4. A test fixture according to claim 1, comprising:
a switching circuit, the switching circuit arranged to electrically couple each electromagnetic coil of the plurality of electromagnetic coils to a signal driving circuit associated with the electronic sensor device.

5. A test fixture according to claim 4, wherein the switching circuit is arranged to electrically couple each electromagnetic coil of the plurality of electromagnetic coils to the signal driving circuit associated with the electronic sensor device in a determined sequence.

6. A test fixture according to claim 4, wherein the switching circuit is arranged to electrically couple two electromagnetic coils of the plurality of electromagnetic coils to the signal driving circuit associated with the electronic sensor device concurrently.

7. A test fixture according to claim 4, comprising:
a plurality of drive signal conduits, each drive signal conduit associated with a different one of the plurality of electromagnetic coils, the plurality of drive signal conduits arranged to facilitate the passage of drive signals to its associated electromagnetic coil.

8. A test fixture according to claim 4, comprising:
at least one wireless transceiver arranged to facilitate the passage of drive signals to the plurality of electromagnetic coils.

9. A test fixture according to claim 1, comprising:
a plurality of support structures physically coupled to the test fixture, at least one electromagnetic coil of the plurality of electromagnetic coils integrated with each support structure.

10. A test fixture according to claim 9, wherein individual ones of the plurality of support structures may be repositioned.

11. An electronic sensor device test fixture, comprising:
an electronic sensor device receiving mechanism arranged to receive an electronic sensor device; and
a plurality of electromagnetic coils arranged in known positions relative to the electronic sensor device receiving mechanism.

12. An electronic sensor device test fixture according to claim 11, wherein the plurality of electromagnetic coils are removably arranged in known positions relative to the electronic sensor device receiving mechanism.

13. An electronic sensor device test fixture according to claim 11, wherein the plurality of electromagnetic coils are permanently arranged in known positions relative to the electronic sensor device receiving mechanism.

14. An electronic sensor device test fixture according to claim 11, wherein the electronic sensor device receiving mechanism includes at least one registration mechanism to align the electronic sensor device.

15. An electronic sensor device test fixture according to claim 11, comprising:
a base unit, the base unit having the electronic sensor device receiving mechanism integrated therein; and
a plurality of support structures affixed to the base unit, at least some of the plurality of support structures having at least some of the plurality of electromagnetic coils integrated therein.

16. A test fixture method, comprising:
coupling an electronic sensor device to a test fixture, the test fixture having a surface and a plurality of electromagnetic coils, each electromagnetic coil of the plurality arranged in a known or knowable three-dimensional position and orientation relative to any point on the surface;
directing at least one drive signal to at least one electromagnetic coil of the plurality of electromagnetic coils to energize the respective at least one electromagnetic coil;
sensing a magnetic field generated about each energized electromagnetic coil; and
generating position and orientation information associated with each energized electromagnetic coil.

17. A test fixture method according to claim 16, comprising:
sequentially directing a plurality of drive signals to various ones of the plurality of electromagnetic coils; and
capturing the generated position and orientation information associated with each energized electromagnetic coil.

18. A test fixture method according to claim 17, comprising:
storing the generated position and orientation information in the electronic sensor device as calibration information.

19. A test fixture method according to claim 16, wherein the magnetic field formed about each energized each coil has characteristics directed by characteristics of the at least one drive signal.

20. A test fixture method according to claim 16, wherein at least two electromagnetic coils of the plurality of electromagnetic coils are concurrently energized.
